(19) United States
(12) Patent Application Publication
(10) Pub. No.: US 2025/0269129 A1
(43) Pub. Date: Aug. 28, 2025

(54) METHOD AND DEVICE FOR MEASURING VOLUMETRIC CAPNOMETRY, OXIMETRY AND FUNCTIONAL RESIDUAL CAPACITY (FRC)

(71) Applicant: Loewenstein Medical Technology S.A., Luxembourg (LU)

(72) Inventor: Peter KREMEIER, Karlsruhe (DE)

(21) Appl. No.: 19/057,110

(22) Filed: Feb. 19, 2025

(30) Foreign Application Priority Data
Feb. 28, 2024 (DE) 10 2024 105 703.3

(51) Int. Cl. A61M 16/00; A61M 16/08; A61M 16/12

(52) U.S. Cl. CPC A61M 16/024; A61M 16/0003; A61M 16/0833; A61M 16/085; A61M 16/0883; A61M 16/12; A61M 2016/0018; A61M 2016/0036; A61M 2202/0007; A61M 2202/0208; A61M 2202/0266; A61M 2205/10; A61M 2205/3303; A61M 2205/3327; A61M 2205/3331; A61M 2205/50; A61M 2230/43

(57) ABSTRACT

A device for measuring metabolic parameters and functional residual capacity is connected to a ventilator. To measure CO₂ and O₂ concentrations, the device takes a sample from a patient's breathing circuit and measures the gas flow rate in the circuit in parallel. The respiratory gas flow rate is measured without a time lag. The CO₂ and O₂ concentration data differ in time by the transport lag in sample delivery from the patient circuit to the CO₂ and O₂ sensors. The processor measures and compensates for transport lags. Time-synchronized CO₂ and O₂ concentration data are time-integrated with the flow rate, and the volumetric amounts of CO₂, O₂/N₂ are calculated. On the basis of these data, metabolic parameters and/or volumetric capnometry function and/or functional residual capacity are calculated.

### CROSS-REFERENCE TO RELATED APPLICATIONS

The present application claims priority under 35 U.S.C. § 119 of German Patent Application No. 102024105703.3, filed Feb. 28, 2024, the entire disclosure of which is expressly incorporated by reference herein.

### BACKGROUND OF THE INVENTION

### 1. Field of the Invention

The present invention relates to the field of monitoring gas exchange parameters, the ventilation mechanics of ventilators and the optimization of the control of ventilators.

### 2. Discussion of Background Information

Volumetric capnometry, volumetric oximetry, metabolic parameters and functional residual capacity (FRC) are all important aspects of respiratory physiology.

Some patents describe various variants of a method of FRC measurement using an indicator gas, for example in documents U.S. Pat. No. 5,540,233 A, US20220257141 A1, US20020052560 A1. The indicator gas used in these patents is helium or sulfur hexafluoride or fluoropropane.

US20020052560 A1 describes a measurement method that is based on apnea episodes. In this measurement method, the indicator gas is the patient's etCO₂. Moreover, the cardiac output is determined using Fick's method.

U.S. Pat. No. 8,371,298 discloses a solution having the following features:
The oxygen sensor is located in the breathing circuit exhalation line;
A slow-reacting oxygen sensor is used to measure the average O₂ concentration in the exhalation line;
The CO₂ concentration is not taken into account, reducing the accuracy of the measurements.

The entire disclosures of the documents mentioned above are expressly incorporated by reference herein.

The present invention relates to the precise monitoring and analysis of respiratory gases during the ventilation of a patient. It would be advantageous to have available a device and a method that:
allow time-synchronized measurement of CO₂ and O₂ concentrations and of the respiratory gas flow,
determine functional residual capacity (FRC) without the use of indicator gases,
calculate metabolic parameters such as VO₂ and VCO₂ and use these to optimize ventilation,
detect artifacts and minimize measurement errors, and
allow simple integration in existing ventilators.

Some advanced ventilators and respiration monitoring systems are able to provide integrated measurements of metabolic parameters and lung volumes. Some of these systems comprise devices that provide real-time data on oxygen consumption, carbon dioxide production and lung volume without the need for the patient to be separated from the ventilator.

However, the correct synchronization of the measurement values is crucial in order to avoid interference and make the results plausible.

The further problem addressed by the present invention is therefore that of synchronizing the measurement values of the total gas flow rate in the patient circuit, the oxygen flow and the carbon dioxide flow over time.

### SUMMARY OF THE INVENTION

The aforementioned problems are solved, inter alia, by a device and a method as set forth in the independent claims. The dependent claims relate to various mutually independent advantageous developments of the present invention, the features of which can be combined freely with one another by a person skilled in the art within the scope of what is technically meaningful. In particular, this also applies beyond the boundaries of the various claim categories.

The invention provides a device for time-synchronized measurement of CO₂ and O₂ fractions in the respiratory gas and respiratory gas flow during ventilation, having an inspiration line, an expiration line and a Y-connector and a line to the patient port, the Y-connector connecting the inspiration line, the expiration line and the line to the patient port, comprising a flow sensor for ascertaining flow data V(t), an O₂ sensor for ascertaining O₂(t) and a CO₂ sensor for ascertaining CO₂(t) and a processor, wherein the processor is configured and designed
to store flow data V(t) and CO₂(t) in the memory,
to determine the inhalation phase IN or exhalation phase EX in the series of measurement values from the flow sensor and from the sensors for CO₂ and for O₂,
to determine the time offset between the measurement values from the flow sensor and from the CO₂ sensor vis-à-vis the O₂ sensor, and
to synchronize the measurement values according to the time offset.

The device comprises a memory for this purpose, or the memory is formed in the ventilator or externally.

The device acquires real-time information about the biologically most important gases—O₂ and CO₂—in the respiratory air. Ascertaining CO₂(t) via the CO₂ sensor, in particular, is helpful in detecting the respiratory phase since the CO₂ fraction typically increases in the exhaled gas.

Possible artifacts such as unwanted leaks can also be uncovered by ascertaining CO₂(t).

According to an embodiment, the processor 9 is configured and designed to use the synchronized concentration functions O₂(t), CO₂(t) and flow V(t) to calculate the volumetric quantities VO₂ and VCO₂.

According to an advantageous embodiment, the processor 9 is configured and designed to use the VO₂ data and VCO₂ data to calculate the metabolic parameters REE and RQ using the Weir equation. The REE and RQ parameters are calculated for each respiratory cycle and averaged over a time interval chosen by the user.

According to a further advantageous embodiment, the processor 9 is configured and designed to use the V(t) data and VCO₂(t) data to create a volumetric capnometry diagram, in which V(t) is on the vertical axis and VCO₂(t) on the horizontal axis.

Advantageously, the processor 9 is configured and designed to use the V(t) data, VO₂(t) data and VCO₂(t) data to calculate the functional residual lung capacity (FRC).

The device may take the form of a part of a ventilator 15 or be an additional module for a ventilator, wherein the ventilator 15 comprises an oxygen mixer 20 in that case.

The processor 9 may be a part of the device or a part of the ventilator 15.

For example, or in a preferred case, the processor 9 is also configured and designed, for an FRC measurement, to periodically modify the oxygen concentration in the inhaled respiratory gas by a predetermined value by means of the oxygen mixer 20 and, by measuring the amount of O₂ and CO₂ in the exhaled respiratory gas, ascertain a function of the amount of nitrogen in the exhaled respiratory gas according to the formula:

N2(t)=Vexp(t)−O₂(t)−CO₂(t);
where:
N2(t)—a function of the amount of nitrogen in the exhaled air;
Vexp(t)—a function of the exhalation flow rate;
O₂(t)—a function of the amount of oxygen in the exhaled air;
CO₂(t)—a function of the amount of carbon dioxide in the exhaled air.

In an embodiment, the processor 9 is configured and designed to determine the FRC according to the following formula:

FRC=(VN1−VN2)/(CN2−CN1)
where
VN1—inspiratory nitrogen volume
VN2—expiratory nitrogen volume
CN1—nitrogen volume fraction in previous exhalation
CN2—fraction of nitrogen volume during the current exhalation.

According to a further embodiment, the processor 9 is configured and designed to perform the calculations after the change of the oxygen concentration in each respiratory cycle.

According to a further advantageous embodiment, the O₂ sensor and the CO₂ sensor are connected via a sampling line to the Y-connection piece in order to guide respiratory gas to the sensors.

By preference, the device comprises a suction device that is configured to guide respiratory gas via the sampling line to the sensors.

Further preferably, the flow sensor is arranged in a line between the Y-connector and the patient.

Hence, it is particularly advantageous that the sampling line is connected on one side to the Y-connection piece and on the other side via a sample dryer, which removes aqueous condensate from the analyzed gas, and the sensors 7 and 8 are arranged downstream thereof.

The invention further provides a method for time-synchronized measurement of CO₂ and O₂ fractions in the respiratory gas and respiratory gas flow during ventilation, having an inspiration line, an expiration line and a Y-connector and a line to the patient port, the Y-connector connecting the inspiration line, the expiration line and the line to the patient port, comprising a flow sensor 2, an O₂ sensor 8 and a CO₂ sensor 7 and a processor 9, wherein the processor 9 is configured and designed to ascertain the respiratory phase inspiration IN or expiration EX from the series of measurement values from the flow sensor 2, from the O₂ sensor 8 and from the CO₂ sensor 7, to determine a time offset of the measurement values from the flow sensor 2, from the O₂ sensor 8 and from the CO₂ sensor 7 to one another from the respective respiratory phase and to synchronize the measurement values according to the time offset.

The invention further provides a method and a device for measuring volumetric capnometry, oximetry and functional residual capacity (FRC) and for optimizing the control of ventilators.

The invention further provides a device for time-synchronized measurement of CO₂ and O₂ fractions in the respiratory gas and respiratory gas flow during ventilation:
having an inspiration line, an expiration line and a Y-connection piece and a line to the patient port, the Y-connection piece connecting the inspiration line, the expiration line and the line to the patient port,
comprising a flow sensor, an O₂ sensor and a CO₂ sensor and a processor, wherein the processor is configured and designed
to ascertain the respiratory phase inspiration IN or expiration EX from the series of measurement values from the flow sensor, from the O₂ sensor and from the CO₂ sensor, to determine a time offset of the measurement values from the flow sensor, the O₂ sensor and the CO₂ sensor to one another from the respective respiratory phase and to synchronize the measurement values according to the time offset.

Specific advantages of the invention preferably include for example
Synchronization of measurement data: The processor automatically detects and compensates for time offsets between flow data, CO₂ data and O₂ data, whereby the measurements are precisely synchronized.
No need for indicator gases: The FRC is calculated by the nitrogen washout method, reducing complexity and facilitating the use in clinical environments.
Automated calculations: Parameters such as REE and RQ are calculated in real time, improving patient metabolism monitoring.
Detection of artifacts: The device automatically identifies leaks or implausible measurement values and signals potential errors.
Integration in ventilators: The device can be used as a module or as part of a ventilator and can easily be retrofitted.

### Device for Synchronized Measurement

A device for synchronized measurement of CO₂ and O₂ fractions in the respiratory gas and respiratory gas flow according to the invention comprises:
a flow sensor (2),
a CO₂ sensor (7),
an O₂ sensor (8),
a sampling line (13) with a sample dryer (12),
a processor (9) which is designed to synchronize the measurement data and calculate volumetric parameters.

According to the invention, FRC determination without indicator gas may be achieved by virtue of the functional residual capacity (FRC) being determined by the nitrogen washout method without the use of indicator gases.

According to the invention, fault detection and artifact management may be achieved by virtue of the processor being capable of detecting leaks and measurement errors and issuing corresponding warnings.

According to the invention, integration in ventilators may be achieved by virtue of being designed as a module or integral constituent part of a ventilator.

According to the invention, volumetric capnometry may be achieved by virtue of the processor creating a volumetric capnometry diagram, wherein the respiratory flow (V) is plotted on the vertical axis and the exhaled CO₂ (VCO₂) is plotted on the horizontal axis.

According to the invention, an automated adjustment of the ventilation may be achieved by virtue of the calculated metabolic parameters (REE, RQ) being used for the automated control of the ventilation.

According to the invention, fast data processing may be achieved by virtue of synchronizing and calculating the parameters in real time.

According to the invention, a modular structure may be achieved by virtue of being constructed in modular fashion and being able to be easily integrated in existing systems.

Measuring CO₂ during ventilation is important in order to monitor respiratory function and gas exchange in the lungs.

The main function of respiration consists of absorbing oxygen and releasing carbon dioxide (CO₂). The CO₂ measurement allows monitoring of the gas exchange in the lungs. Excessively high or low CO₂ content in the respiratory air flow may indicate problems in the gas exchange.

The CO₂ measurement provides information about the effectiveness of ventilation, i.e., how well the air enters the lungs and how efficiently the CO₂ is removed from the body. Inadequate ventilation may lead to an increase in CO₂ content in the blood, which is referred to as hypercapnia.

Measuring CO₂ is particularly important when monitoring patients who are mechanically ventilated. It helps with detecting respiratory disorders such as hypoventilation or hyperventilation and making appropriate adjustments to the ventilation.

For patients who are sedated or anesthetized, the CO₂ measurement may contribute to monitoring the depth of sedation and ensuring that the patient is adequately ventilated.

Monitoring the CO₂ content in the respiratory air flow may contribute to a timely detection of respiratory failure. This is particularly important in critical medical situations where immediate intervention may be required.

Overall, the CO₂ measurement during ventilation allows precise monitoring of the respiratory function.

In particular, what is important with respect to the ventilator is recognizing that the functional state of the ventilated lungs, which changes from one breath of the artificially ventilated patient to the next, is incorporated in the regulation of the ventilation settings. A new dimension in ventilation therapy is achieved by the simultaneous incorporation of both technical and physiological characteristic variables, in the form of technical ventilation parameters on the one hand and in the form of patient-specific physiological parameters, for example O₂ values and/or CO₂ values and/or FRC values, on the other, in the regulation of the ventilator. This procedure allows safe control of ventilation therapy.

The proposed ventilator is able to determine the state of the lung in automated fashion via repeated measurement of O₂ and/or CO₂ and/or FRC and able to automatically adjust the technical ventilation parameters at regular intervals via the proposed control in such a way that the patient is subjected to artificial ventilation which is successful and also gentle.

According to the invention, the solution finds use, for example, in a ventilator with open or closed loop control and feedback control algorithms, in order to automatically adjust the settings of the ventilator on the basis of the physiological measurement values.

The ventilator is geared toward optimizing ventilation and gas exchange parameters in order to improve patient outcomes and reduce the risk of ventilator-induced lung damage.

Volumetric capnometry, volumetric oximetry, metabolic parameters and functional residual capacity (FRC) are all important aspects of respiratory physiology. The following illustrates how each of these parameters can be calculated, according to the invention or in modified fashion, and used.

Volumetric capnometry is a technique for measuring the volume of carbon dioxide (CO₂) exhaled by the lungs over time. The most important parameters of volumetric capnometry are the capnogram and volumetric capnogram, a graphical representation of the CO₂ concentration and CO₂ amount over time during the respiratory cycle.

The area under the volumetric capnogram curve may provide information about the total amount of exhaled CO₂, which is related to the efficiency of ventilation. The CO₂ concentration in the exhaled air is the most important parameter for monitoring ventilation therapy.

The area under the volumetric capnogram curve is usually measured using special software or equipment. This area represents the total volume of carbon dioxide (CO₂) exhaled during the breathing cycle. Capnogram curve integration is often automated in capnography devices.

Further preferably, the measurement of carbon dioxide values can be implemented by means of volumetric capnography and directly determining parameters representing the CO₂ gas exchange in the lung of the patient, for example end-expiratory CO₂ partial pressure in the exhaled gas mixture, alveolar CO₂ partial pressure or volume of CO₂ eliminated in a single breath of the patient.

Capnography is therefore a suitable means for determining and also graphically representing the amount or fraction of expiratory carbon dioxide, or expiratory CO₂ for short. The CO₂ kinetics of mechanically ventilated patients are represented in a non-invasive way and in real time. Volumetric capnography in particular is a suitable means for the clinical monitoring of mechanically ventilated patients.

By means of volumetric capnography, it is possible to measure the CO₂ concentration in respiratory gases over the respiratory cycle. For example, the CO₂ concentration is calculated from the absorption of infrared light according to the Beer-Lambert law and usually expressed as a partial pressure in units of mmHg. The graphical representation of the elimination of CO₂ during respiration is referred to as a capnogram and the corresponding measurement device is referred to as a capnograph.

So-called sidestream capnographs are devices which aspirate a respiratory gas sample from the airway opening of the ventilated patient and transport it via a tube system to sensors distant from the aspiration point, in order to measure the CO₂ contained in said sample. The sidestream method can also be used for non-intubated patients via a nasal cannula with a CO₂ aspiration line. In addition, the sidestream method has the great advantage that no additional measurement cuvette is required. This significantly reduces the dead space in the patient tubing system and also reduces the weight of the ventilation tube at the patient end. The CO₂ measurement takes place with a slight time lag in the sidestream method and is therefore slightly slower than the mainstream measurement.

Then again, mainstream capnographs are devices in which the CO₂ sensors, and in some cases the flow sensors as well, are located in a measuring head on the Y-piece of the ventilation tube, and so in situ measurements may be performed near the airway opening using this method. Mainstream capnographs do not cause volume loss and measure the total respiratory gas volume. The additional measurement cuvette between the patient valve and the tube results in an increased dead space volume in the mainstream method.

There is no time lag during the measurement, and the total amount of air can be taken into account. Both sidestream capnographs and mainstream capnographs may be used in the context of the present invention, wherein sidestream capnographs are preferably used. Capnography describes the continuous measurement of end-tidal CO₂ (etCO₂) in exhaled gas, implemented by way of infrared spectroscopy in the sidestream method.

As a rule, capnography is classified according to its graphical representation, with time-based or standard capnography being the most common type of capnogram. The CO₂ concentration is plotted over time in this case. By contrast, volume-based or volumetric capnography represents the amount of carbon dioxide eliminated in one breath, in relation to the exhaled volume of the breath. Unlike standard capnography, volumetric capnography is advantageously able to acquire volumetric parameters that are of clinical importance. These include the pulmonary elimination of CO₂, dead space and alveolar ventilation. Both time-based or standard capnography and volume-based or volumetric capnography may be used in the context of the present invention.

In an analogous manner to the previously discussed parameter directly representing the CO₂ gas exchange in the lung of the patient, it is advantageously possible in an alternative to that or in addition for parameters directly representing the oxygen gas exchange (in short: O₂ gas exchange) in the lung of the patient to be used as patient-specific physiological parameters.

According to a further aspect of the teaching, a ventilator for artificial ventilation of a patient is proposed. The proposed ventilator comprises: a controller configured to define at least one technical ventilation parameter, wherein the patient is ventilated on the basis of the technical ventilation parameter, wherein the at least one technical ventilation parameter corresponds to at least one of the ventilation parameters of respiratory minute volume, tidal volume, respiratory rate, positive end-expiratory pressure or inspiratory oxygen concentration provided by the ventilator; and a regulation unit which is in communication with the measurement device and with the controller. The control unit is configured such that it adjusts the at least one technical respiration parameter on the basis of the repeating measurement of O₂ and/or CO₂ and/or FRC.

Fast-reacting oxygen sensors with a performance in the same order of magnitude as that of the capnograph should be used to perform volumetric oxygraphy. Failure to do so will result in measurement errors due to distortion of the shape of volumetric oxygraphy.

The O₂ and CO₂ concentrations should be measured in succession in the same gas sample.

Metabolic parameters associated with respiratory physiology often refer to parameters related to oxygen consumption (VO₂) and carbon dioxide production (VCO₂).

The respiratory quotient (RQ) is such a metabolic parameter and calculated as the ratio of VCO₂ to VO₂. RQ=VCO₂/VO₂. The RQ provides information about the substrate that is metabolized. For example, RQ values of 1.0 indicate a carbohydrate metabolism, while values below 1.0 indicate a fat metabolism.

During the measurement, the inhaled and exhaled air is examined for its oxygen and carbon dioxide concentrations. Appropriate devices automatically calculate VO₂ and VCO₂ on the basis of the gas concentrations and airflow. This technique provides valuable information about the metabolism and gas exchange of the patient's airways.

Functional residual capacity (FRC) is the volume of air in the lungs at the end of passive exhalation. It represents the equilibrium between the elastic recoil of the lungs and the recoil of the chest wall to the outside. FRC can be measured using various techniques, including body plethysmography, helium dilution, or nitrogen washout.

In this technique, the air in the lungs is replaced with a known concentration of nitrogen. The initial lung volume (V1) is measured before the nitrogen is introduced. The nitrogen concentration (F1) and the final concentration of nitrogen (Fi) are measured as soon as equilibrium is reached. The final volume of the lung (V2) is subsequently measured.

A formula for calculating FRC using the nitrogen washout technique is FRC=V1+(F1/Fi)*V2−V1), where V1 is the initial volume of the lung, F1 is the initial concentration of nitrogen, Fi is the final concentration of nitrogen and V2 is the final volume of the lung.

According to a first advantageous embodiment of the ventilator, provision is made for the ventilator to comprise:
a gas flow sensor arranged on the Y-connection piece,
a sidestream capnograph,
an oximeter for the sideward flow.

According to a second advantageous embodiment of the ventilator, provision is made for the ventilator to comprise:
a sidestream capnograph
a sidestream oximeter.

The device does not comprise a gas flow sensor at the Y-connection piece but uses the flow data obtained from the integrated flow sensors.

The invention provides a method and a device 14 for measuring metabolic parameters and the functional residual capacity. To measure the CO₂ and O₂ concentrations, the device takes a sample from the patient's breathing circuit and measures the gas flow rate in the patient's circuit in parallel. The respiratory gas flow rate is measured without a time lag. The oxygen and carbon dioxide concentration data differ in time by the transport lag in sample delivery from the patient circuit to the CO₂ and O₂ sensors. The processor measures and compensates for transport lags T1 and T2 at characteristic points. Time-synchronized CO₂ and O₂ concentration data are time-integrated with the flow rate, and the volumetric amounts of CO₂, O₂/N₂ are calculated. On the basis of these data, metabolic parameters (REE, RQ) and/or the volumetric capnometry function and/or the functional residual capacity (FRC) are calculated using the nitrogen washout method, for example. The CO₂ data are also used to detect artifacts and exclude measurement errors, as well as to exclude the CO₂ volume from the leached nitrogen.

The device for volumetric capnometry, metabolic parameters and functional residual capacity (hereinafter referred to as the device) is provided for use in conjunction with ventilators or as an integral constituent part of ventilators. The device comprises a processor that calculates the parameters of volumetric capnometry and of the metabolism and the value of functional residual capacity. For example, these data are transmitted to the ventilator for display purposes.

According to the invention, various parameters of the gas exchange in the lung of the patient, metabolic parameters and respiratory mechanics, which are normally measured by several devices, are determined and reliable algorithms for measuring these parameters are provided. The invention offers an innovative solution for the precise monitoring and analysis of respiratory gases during ventilation. It automatically synchronizes measurement data, calculates metabolic parameters in real time, detects artifacts and improves ventilation control by way of its easy integration in modern ventilators. The absence of indicator gases simplifies the application and increases reliability.

### BRIEF DESCRIPTION OF THE DRAWINGS

In the accompanying drawings,

FIG. 1 shows a schematic illustration of the device and ventilator.

FIG. 2 shows a detailed, schematic illustration of the device.

FIG. 3 shows three graphs that show time-curves of flow, oxygen and carbon dioxide values.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

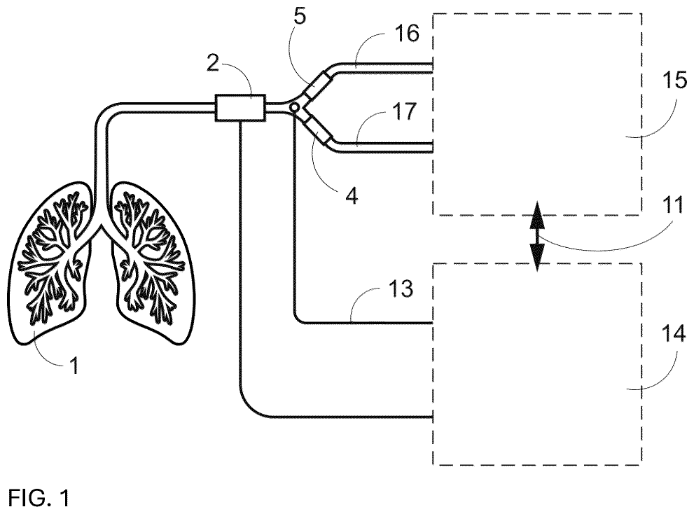

FIG. 1 shows a schematic overview of the equipment consisting of the device and the ventilator. It shows the device 14 as a module of a ventilator 15. The inspiration line 17 and expiration line 16 are connected to the patient via the Y-connector 3. The flow sensor 2 is situated between the Y-connector and the patient port 18. Samples are taken via the sampling line 13 and guided through the sample dryer 12.

The device 14 and the ventilator 15 form a complete system that is connected to the patient 1. The breathing circuit of the ventilator 15 consists of an inspiration line 17, an expiration line 16 and a Y-connection piece 3. The ventilator breathing circuit is connected to the patient 1 via the Y-connection piece 3.

Y-connection piece 3: During the inspiration phase, fresh gas is supplied to the lung of the patient 1 via the inspiration line 17 and the port 4 of the Y-connection piece. During the expiration phase, the gas leaves the lung of the patient 1 to the surroundings via the port 5 of the Y-connection piece, the exhalation line 16 and the ventilator exhalation valve (not shown). Depending on the ventilation mode, some of the inspiration flow may be guided directly from the inspiration line to the expiration line in order to provide the support flow required for the function of the inspiration trigger.

The device 14 is connected to the breathing circuit of the ventilator (Y-connection piece 3) and to the ventilator control system via the output interface 11, as shown in FIG. 1.

The flow sensor 2 of the device is connected between the Y-connection piece 3 and the patient 1. The sampling line 13 of the device 14 is connected on one side to the Y-connection piece 3 and on the other side to the device 14 via a sample dryer 12, which draws aqueous condensate from the analyzed gas.

The sensor 2 is not connected should the ventilator comprise a through-flow sensor, and the through-flow data are transmitted from the ventilator to the device 14 via the output interface 11 in that case.

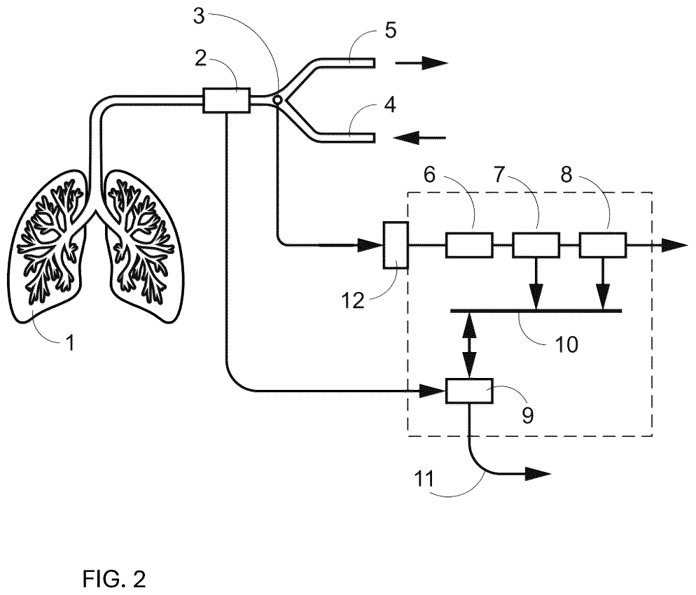

FIG. 2 provides a schematic basis for the following description of the functionality of the device. It represents the internal structure of the device, including micro-compressor 6, which aspirates the samples, and the sensors 7 (CO₂) and 8 (O₂). The processor 9 is connected to a memory and synchronizes the data streams.

The flow sensor 2 measures the gas flow during the inspiration and expiration phases. The flow rate data during the inspiration phase are integrated, and the volume is calculated, as is the expiration volume. The leakage rate is calculated should the inspiratory and expiratory volumes differ.

The sampling line 13 of the device 14 is connected to the sample dehumidification device 12. The sample is aspirated from the patient circuit using the micro-compressor 6. The sample passes through the sample dehumidifier 12 and the dehydrated gas is guided to the carbon dioxide concentration sensor 7 (capnograph) and subsequently on to the oxygen concentration sensor 8.

The gas sample passes through the sampling line 13 in a time of for example 0.2-3 seconds, depending on the length of the sampling line. During this transport time, the CO₂ concentration data of the sensor 7 lag behind the flow rate data. The O₂ data of the sensor 8 are ascertained at a later time than the through-flow data of the sensor 2 and also at a later time than the CO₂ data of the sensor 7 since the O₂ sensor 8 is connected downstream of the CO₂ sensor 7.

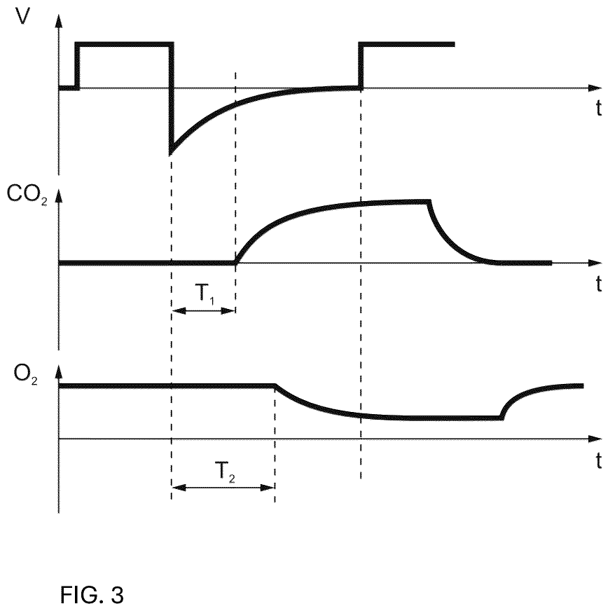

FIG. 3 shows a time-curve of the measurement values of flow, oxygen and carbon dioxide during the ventilation of a patient. FIG. 3 shows the changes of flow (V), CO₂(CO₂(t)) and O₂(O₂(t)) over time. The offsets T1 and T2 are compensated for in an automated manner.

FIG. 3 shows the data from the flow sensor 2 in track 22. These are integrated in order to ascertain the volume V. Alternating phases of inspiration In and expiration Ex over time t can be identified in the volume curve.

The data from the CO₂ sensor 7 can be identified in track 27. Alternating phases of inspiration In and expiration Ex over time t can be identified from the curve. Compared to the times of inspiration In and expiration Ex from track 22, a time offset T1 that relates to the start of expiration Ex can be identified.

The data from the O₂ sensor 8 can be identified in track 28. Alternating phases of inspiration In and expiration Ex over time t can be identified from the curve. Compared to the times of inspiration In and expiration Ex from track 22, a time offset T2 that relates to the start of expiration Ex can be identified.

The time offset results from the time-lagged transport of the gas from the Y-connection piece 3 via the sampling line 13 to the device 14. Due to the transport lag, the CO₂ and O₂ concentration data are available later than the flow data. Due to the transport lag, the CO₂ and O₂ concentration data and flow V data cannot be processed together without a synchronization in time. The values of the T1 and T2 time lags must be determined for synchronization purposes.

To this end, the characteristic points of the start and end of inspiration and expiration are determined on curves 27, 28 of CO₂ and O₂ concentrations. Determination is preferably automated using stored algorithms that identify the respiratory phase from the shape of the curves. The determination is also based on the insight that, during expiration, the CO₂ fraction in the respiratory gas is increased and the O₂ fraction is reduced.

Then, the flow curve 22 is delayed by the value of T2 and the CO₂ concentration curve 27 by the value of T2-T1, whereby the time synchronization of all curves is achieved.

In this exemplary embodiment, the sensors are arranged in succession in the flow direction of the gas; first the CO₂ sensor and then the O₂ sensor. The O₂ sensor is the one that has the greatest time lag. Therefore, the flow signal should be delayed by time T2 and the CO₂ signal by a shorter time T2-T1.

The synchronized signals from the CO₂ and O₂ sensors and of the flow velocity V are transmitted to the processor 9 (see FIG. 2), where the parameters are calculated on the basis of working algorithms and can be transmitted via the external interface 11 to for example the ventilator for display on the monitor.

The device also has algorithms for determining functional residual capacity (FRC), metabolic parameters and volumetric capnometry.

The sequence of exemplary steps for ascertaining the FRC and the synchronized concentration functions O₂(t), CO₂(t) and flow V(t) is as follows:

The device can be connected to the ventilator's data bus, or it is a constituent part of the ventilator. The device is also connected to the breathing circuit of the ventilator at the Y-connection piece in order to take a sample for analysis. The device uses the flow sensor that is connected to the breathing circuit of the ventilator in the region of the Y-connection piece. The device itself does not comprise a flow sensor and may use flow data from the ventilator, which for example are transmitted via the data bus.

For example, the device itself does not comprise a through-flow sensor and can use through-flow data, which for example are received from a ventilator and transmitted to the device.

The device takes a gas sample in order to measure the oxygen concentration O₂ and the carbon dioxide concentration CO₂. The device comprises a time synchronization algorithm that compensates for the time lag of the gas sample with respect to the flow velocity function V(t). The synchronized concentration functions O₂(t), CO₂(t) and flow V(t) are used to calculate the volumetric quantities VO₂ and VCO₂.

The VO₂ and VCO₂ data are optionally also used to calculate the metabolic parameters REE and RQ using the Weir equation.

The V(t) and VCO₂(t) data are used to create a volumetric capnometry diagram, with V(t) on the vertical axis and VCO₂(t) on the horizontal axis. The V(t) data, VO₂(t) data and VCO₂(t) data are used to calculate functional residual lung capacity (FRC).

The calculated parameters are for example output via the data bus of the ventilator or transmitted to the ventilator and may be displayed and/or stored there or additionally used for ventilation regulation.

The patient is ventilated using a ventilator connected to the patient via a Y-connection piece. The Y-connection piece is connected to an inspiration line for fresh respiratory gas, which is supplied by the ventilator, and an expiration line for removing the exhaled gas from the lung of the patient. The fresh breathing gas is a mixture of air fractions, oxygen and water vapor. The exhaled gas consists of carbon dioxide, air, oxygen and water vapor fractions.

The gas for the relevant device for measuring gas concentrations is taken from the Y-connection piece. The gas flow rate is also measured at this point. This allows not only the concentration but also the amount of oxygen and carbon dioxide during inhalation and exhalation to be calculated. If the water vapor concentration is assumed to be the same during inspiration and expiration, then the residual gas fraction is nitrogen.

Calculating the amount of gas in the region of Y-connection piece 3 eliminates errors on account of the support flow and potential gas leaks in the circuit. The support flow in the ventilator is normally required to actuate the inspiratory trigger and flows directly from the inspiration line to the expiration line.

Placement of the flow sensor in the vicinity of the Y-connector allows the leak to be detected and the size of the leak to be estimated and allows the necessary adjustments to be made to the calculation results. Alternatively, in the event of a large leak, this allows a warning to be issued that the calculation results are unreliable. The carbon dioxide measurement also avoids FRC estimation errors on account of the presence of CO₂ in the breathing circuit. To reduce errors, the dynamic properties of the oxygen and carbon dioxide sensors must be high enough so as not to distort the shape of the inspiratory and expiratory gas fraction streams.

The measurement of the carbon dioxide concentration during exhalation allows assessment of the characteristics of the inspiration and expiration cycle and assessment of the presence of artifacts in a given cycle and allows a warning to be issued regarding possible calculation errors in the event of a large number of artifacts.

In the FRC determination by nitrogen washout, the patient is briefly offered an increased oxygen concentration, for example 100 vol. %, or some of the nitrogen is replaced by an inert gas in the event of an unchanging oxygen concentration. In contrast to nitrogen washout using inert gases, no additional gas is required for a ventilator in the event of nitrogen washout with the aid of an increased oxygen concentration because oxygen and normal respiratory air are normally available there.

For the FRC measurement, the oxygen concentration in the inhaled gas is periodically modified by a predetermined value. A respiratory gas mixer is provided to this end; in particular, it is able to supply 100% oxygen to the respiratory gas and thus able to specify virtually any desired oxygen concentration in the respiratory gas. The respiratory gas mixer 20 can increase and decrease the oxygen concentration in the inhaled gas.

The oxygen concentration in the exhaled air changes uniformly and with a time lag due to the inertia of the O₂ concentration change in the functional residual capacity. The law of concentration change is almost exponential. The process of stabilizing the O₂ concentration is estimated using the EtO₂ and EtCO₂ values at the end of exhalation.

By measuring the amount of O₂ and CO₂ in the exhaled air, it is also possible to ascertain a function of the amount of nitrogen in the exhaled air: This is based on the fact that added oxygen displaces nitrogen. 100% O₂ ventilation displaces nitrogen from the lung. In air, the nitrogen fraction is always 79%. Oxygen can replace nitrogen in breathing circuits or in respiratory gases. The nitrogen fraction can be calculated on the basis of these conditions.

N2(t)=Vexp(t)−O₂(t)−CO₂(t);
where:
N2(t)—a function of the amount of nitrogen in the exhaled respiratory gas;
Vexp (t)—a function of the exhalation flow rate;
O₂(t)—a function of the amount of oxygen in the exhaled respiratory gas;
CO₂(t)—a function of the amount of carbon dioxide in the exhaled respiratory gas.

The curves are used for metabolic calculations and volumetric capnometry.

The FRC calculation algorithm provides the end-expiratory lung volume:

FRC=(VN1−VN2)/(CN2−CN1)
where
VN1—inspiratory nitrogen volume
VN2—expiratory nitrogen volume
CN1—nitrogen volume fraction in previous exhalation
CN2—fraction of nitrogen volume during the current exhalation.

For example, the calculation is performed in each respiratory cycle following the change in oxygen concentration. Averaging is performed over several cycles.

What is claimed is:

1. A device for time-synchronized measurement of CO₂ and O₂ fractions in the respiratory gas and respiratory gas flow during ventilation, wherein the device comprises
an inspiration line, an expiration line and a Y-connection piece and a line to a patient port, the Y-connection piece connecting the inspiration line, the expiration line and the line to the patient port,
a flow sensor for ascertaining flow data V(t), an O₂ sensor for ascertaining O₂(t), a CO₂ sensor for ascertaining CO₂(t) and a processor, the processor being configured
to store flow data V(t) and CO₂(t) in a memory,
to determine an inhalation phase IN or exhalation phase EX in a series of measurement values from the flow sensor and from the sensors for CO₂ and for O₂,
to determine a time offset between the measurement values from the flow sensor and from the CO₂ sensor vis-à-vis the O₂ sensor, and
to synchronize the measurement values according to the time offset.

2. The device of claim 1, wherein the processor is configured to use synchronized concentration functions O₂(t), CO₂(t) and flow V(t) to calculate volumetric quantities VO₂ and VCO₂.

3. The device of claim 1, wherein the processor is configured to use the VO₂ data and VCO₂ data to calculate metabolic parameters REE and RQ using the Weir equation, REE and RQ being calculated for each respiratory cycle and averaged over a time interval chosen by a user.

4. The device of claim 1, wherein the processor is configured to use the V(t) data and VCO₂(t) data to create a volumetric capnometry diagram, in which V(t) is on a vertical axis and VCO₂(t) is on a horizontal axis.

5. The device of claim 1, wherein the processor is configured to use the V(t) data, VO₂(t) data and VCO₂(t) data to calculate a functional residual lung capacity (FRC).

6. The device of claim 1, wherein the device is part of a ventilator or an additional module for a ventilator, the ventilator comprising an oxygen mixer.

7. The device of claim 1, wherein the processor is a part of the device or a part of the ventilator.

8. The device of claim 1, wherein the processor is configured for an functional residual lung capacity (FRC) measurement, to periodically modify an oxygen concentration in an inhaled respiratory gas by a predetermined value by an oxygen mixer and, by measuring an amount of O₂ and CO₂ in an exhaled respiratory gas, ascertain a function of an amount of nitrogen in the exhaled respiratory gas according to the formula
N2(t)=Vexp(t)−O₂(t)−CO₂(t);
where:
N2(t)—a function of the amount of nitrogen in the exhaled respiratory gas;
Vexp (t)—a function of exhalation flow rate;
O₂(t)—a function of the amount of oxygen in the exhaled respiratory gas;
CO₂(t)—a function of the amount of carbon dioxide in the exhaled respiratory gas.

9. The device of claim 1, wherein the processor is configured to determine the functional residual lung capacity (FRC) according to the following formula:
FRC=(VN1−VN2)/(CN2−CN1)
where
VN1—inspiratory nitrogen volume
VN2—expiratory nitrogen volume
CN1—nitrogen volume fraction in previous exhalation
CN2—fraction of nitrogen volume during current exhalation.

10. The device of claim 1, wherein the processor is configured to perform calculations after a change of an oxygen concentration in each respiratory cycle.

11. The device of claim 1, wherein the O₂ sensor and the CO₂ sensor are connected via a sampling line to the Y-connection piece in order to guide respiratory gas to the sensors.

12. The device of claim 1, wherein the device further comprises a suction device that is configured to guide respiratory gas via the sampling line to the sensors.

13. The device of claim 1, wherein the flow sensor is arranged in a line between the Y-connection piece and a patient.

14. The device of claim 1, wherein the sampling line is connected on one side to the Y-connection piece and on the other side via a sample dryer, which removes aqueous condensate from analyzed gas, and the sensors for CO₂ and for O₂ are arranged downstream thereof.

15. A method for time-synchronized measurement of CO₂ and O₂ fractions in the respiratory gas and respiratory gas flow during ventilation,
having an inspiration line, an expiration line, a Y-connection piece and a line to a patient port, the Y-connection piece connecting the inspiration line, the expiration line and the line to the patient port,
comprising a flow sensor, an O₂ sensor, a CO₂ sensor and a processor, the processor being configured to ascertain respiratory phase inspiration IN or respiratory phase expiration EX from a series of measurement values from the flow sensor, from the O₂ sensor and from the CO₂ sensor to determine a time offset of the measurement values from the flow sensor, the O₂ sensor and the CO₂ sensor to one another from the respective respiratory phase and to synchronize the measurement values according to the time offset.
